(19) United States
(12) Plant Patent Application Publication
(10) Pub. No.: US 2026/0060155 P1
(43) Pub. Date: Feb. 26, 2026

(54) Prunus rootstock named 'RVL-4'

(50) ;

(71) Applicants: Gennady Eremin, Krymsk (RU); Oxana Eremin, Krymsk (RU)

(72) Inventors: Gennady Eremin, Krymsk (RU); Oxana Eremin, Krymsk (RU)

(21) Appl. No.: 19/301,524

(22) Filed: Aug. 15, 2025

Related U.S. Application Data
(60) Provisional application No. 63/686,300, filed on Aug. 23, 2024.

(51) Int. Cl. A01H 6/74; A01H 5/06; A01H 5/12

(52) U.S. Cl. CPC A01H 6/7445; A01H 5/06; A01H 5/12
USPC PLT/183

(57) ABSTRACT

A new and distinct cultivar of Prunus rootstock tree named ‘RVL-4’ is disclosed, characterized by high precocity and mildew resistance. ‘RVL-4’ is suitable for use as a rootstock upon which commercial fruit bearing sweet cherry varieties may be budded or grafted. The new variety is a Prunus vulgaris x maacki x lannesiana, typically produced for rootstock.

Latin name of the genus and species: Prunus vulgaris x maacki x lannesiana

Variety denomination: ‘RVL-4’

### BACKGROUND OF THE INVENTION

The new cultivar is a product of a planned breeding program. The new variety resulted from a cross pollination breeding program conducted in Krymsk, Russia The seed parent is the unpatented variety Prunus vulgaris x maacki ‘Rubin’. The pollen parent is the unpatented variety Prunus lannesiana ‘L2’ The cross pollination resulting in the new cultivar occurred during 1995. The new variety was first selected in 2004, by the inventors in Krymsk, Russia.

Asexual reproduction of the new cultivar ‘RVL-4’ by vegetative cuttings was first in performed in 2004 at a nursery in Krymsk, Russia and after 15 years of confidential testing has shown that the unique features of this cultivar are stable and reproduced true to type on successive generations.

### SUMMARY OF THE INVENTION

The cultivar ‘RVL-4’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘RVL-4’. These characteristics in combination distinguish ‘RVL-4’ as a new and distinct Prunus cultivar:
1. Suitability for use as a rootstock upon which commercial fruit bearing sweet cherry varieties may be budded or grafted.
2. High precocity.
3. Mildew resistance.
4. Induces healthy lateral branch development.

### COMMERCIAL COMPARISON

Plants of the new cultivar ‘RVL-4’ are similar to plants of the known commercial variety ‘L5’, unpatented in most horticultural characteristics, however, plants of the new cultivar ‘RVL-4’ differ in the following:
1. Increased precocity by one year.
2. Later flowering time by approximately 2 weeks.
3. Narrow foliage.

### BRIEF DESCRIPTION OF THE PHOTOGRAPHS

The accompanying photographs illustrate some typical characteristics of ‘RVL-4’. The photograph weas taken using conventional techniques and although colors may appear different from actual colors due to light reflectance it is as accurate as possible by conventional photographic techniques.

FIG. 1 shows an approximately 3-year old tree of ‘RVL-4’ which has been cut back to generate new growth for propagation.

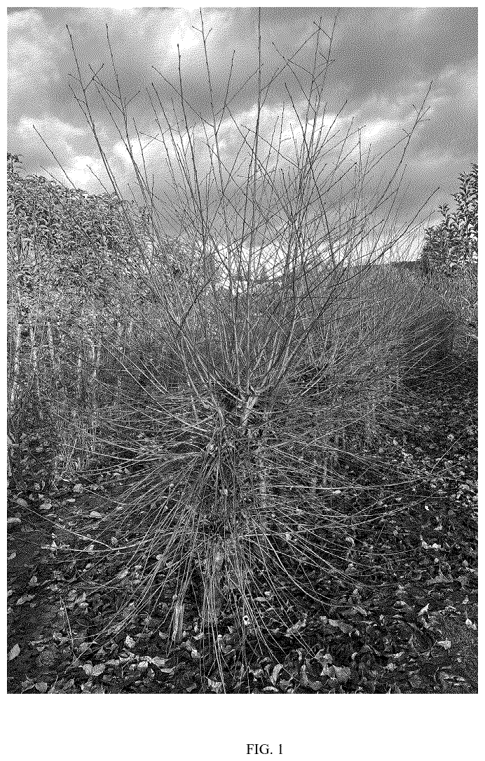

FIG. 2 is a close-up view of the new growth in FIG. 1.

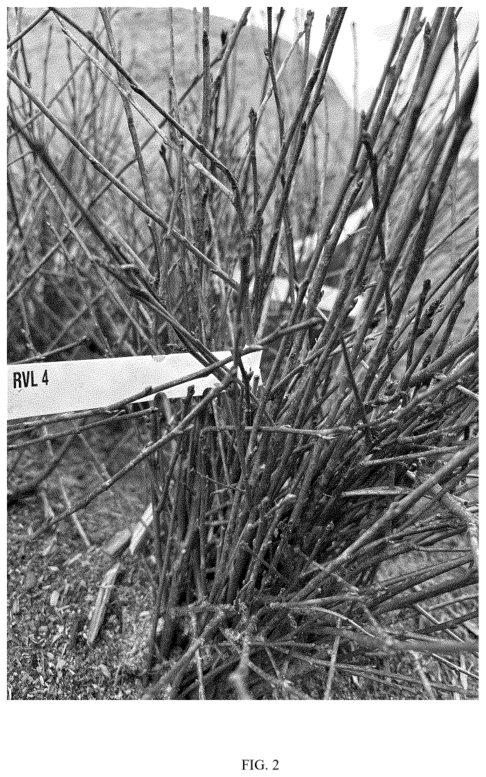

FIG. 3 shows a leafed out tree of ‘RVL-4’ at about 1.5 years of age.

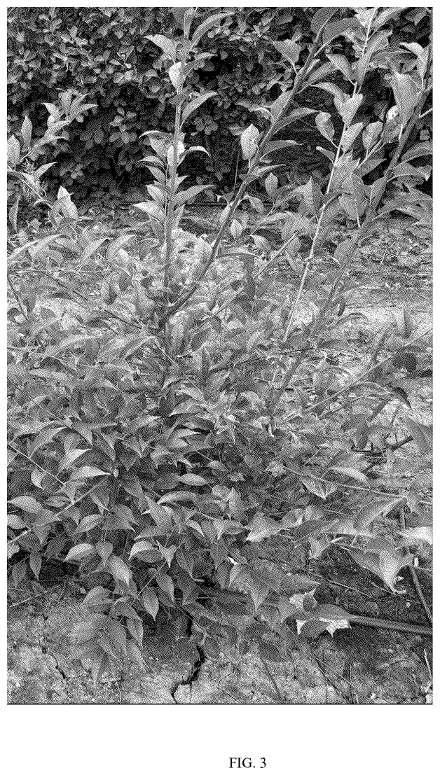

### DETAILED BOTANICAL DESCRIPTION

In the following description, color references are made to the Royal Horticultural Society Colour Chart 2007 except where general terms of ordinary dictionary significance are used. The following observations and measurements describe ‘RVL-4’ plants grown outdoors in Krymsk, Russia. Measurements and numerical values represent averages of typical plant types.
Botanical classification: Prunus ‘RVL-4’

### ROOTS

Root description: Typical Prunus root, coarse, semi-woody roots, typically colored near RHS Grey-Brown 199A.

### PLANT

Growth habit: Spreading deciduous tree.
Branching characteristics: Not typically grown for above ground characteristics.
One-year old shoot internode length: 4.2 cm.
   Texture.—Lenticels present.
   Color.—Striated near Grey-Brown 199A with Greyed-Orange N167A. Immature growth is colored near Greyed-Purple 187A with Greyed-Orange 177D dots.
Vegetative Buds: Approximately 6 mm long and 4 mm wide. Colored near Yellow-Green 8 N144C, heavily flushed Greyed-Purple 187A.
Wood color: Greyed-Orange 165A

### FOLIAGE

Leaf:
   Arrangement.—Alternate.
   Shape.—Narrow elliptic.
   Average length.—8.5 cm.
   Average width.—3.7 cm.
   Attachment.—Stalked.
   Apex.—Acute.
   Base.—Blunt rounded.
   Margin.—Serrate and crenate. Shallow.
   Texture of top surface.—Glabrous, somewhat leathery.
   Texture of bottom surface.—Glabrous.
   Pubescence.—No leaf pubescence.
   Aspect.—Flat to slightly undulating along margins.
   Color.—Mature foliage upper side: Yellow-Green 147A Mature foliage lower side: Yellow-Green 147B.
   Leaf petiole.—Length: 1.5 cm. Diameter: 0.3 cm. Color: Near RHS Greyed-Orange 177A Texture: Slightly pubescent.
   Stipule.—not present.
   Nectaries.—2 round, green nectaries present at base of leaf balde.

### OTHER CHARACTERISTICS

Disease/pest resistance: Neither resistance nor susceptibility to pathogens and pests common to Prunus have been observed.
Temperature tolerance: The new variety is suitable for growing in USDA zones 5 through 9.

What is claimed is:

1. A new and distinct cultivar of Prunus tree named ‘RVL-4’ as herein illustrated and described.
